(19) United States
(12) Patent Application Publication
(10) Pub. No.: US 2012/0010548 A1
(43) Pub. Date: Jan. 12, 2012

(54) Knee Brace to Limit Rotation Between the Femur and Tibia

(76) Inventors: Sara A. Scholtes, Missoula, MT (US); Katherine Damico, Avon, OH (US)

(21) Appl. No.: 13/179,397

(22) Filed: Jul. 8, 2011

Related U.S. Application Data
(60) Provisional application No. 61/362,354, filed on Jul. 8, 2010.

(51) Int. Cl. A61F 5/00

(52) U.S. Cl. 602/26

(57) ABSTRACT

An upper anchor attached to the thigh of a wearer and a lower anchor attached to the calf secure a strap across the knee to allow rotational orthosis control of the tibia and fibula to limit or decrease excessive rotation, and correct resting alignment. The anchors adhere to the skin allowing the strap to be tightened to correct the mal-rotation and to control the corrected position of these two bones thus preventing mal-ration and its associated pain.

### CROSS-REFERENCE TO RELATED APPLICATIONS

This application claims benefit of U.S. Provisional Application No. 61/362,354, filed Jul. 8, 2010, the disclosure of which is hereby incorporated by reference in its entirety including all figures, tables and drawings.

### STATEMENT REGARDING FEDERALLY SPONSORED RESEARCH OR DEVELOPMENT

Not applicable.

### REFERENCE TO SEQUENCE LISTING, A TABLE, OR A COMPUTER PROGRAM LISTING COMPACT DISC APPENDIX

Not applicable.

### BACKGROUND OF THE INVENTION

The knee is a complex anatomical structure of joints, cartilages and bones designed to move primarily in one plane of motion to bend and straighten the knee and thus the leg. Medial/lateral rotation must also occur in a normally functioning knee. If the knee is mechanically functioning properly, an otherwise healthy knee should be pain free. The knee, however, is a commonly injured joint in a variety of different populations. Both acute injury and chronic injury can result in knee pain caused primarily by excessive rotation resulting in mal-alignment of the knee joint's anatomical structures. In particular, people often complain of anterior knee pain, thought to occur due to mal-alignment between the patella and underlying femur. One mechanism contributing to this mal-alignment is excessive or abnormal rotation between the femur and the tibia. The patella is attached to the femur and the tibia through tendons and ligaments, if rotation occurs between the femur and tibia, there is potential for mal-alignment between the patella and the femur. This mal-alignment may result in different pressure points between the patella and femur, eventually resulting in various degrees of pain. Additionally, rotation between the femur and the tibia could result in pain in other soft tissue structures of the knee such as muscles and ligaments. Typical intervention to assist with anterior knee pain thought to be related to mal-alignment between the patella and femur has been to tape or brace the patella back into better alignment. If mal-alignment of the femur and tibia exists, however, then it may be more beneficial to decrease or eliminate rotation between the tibia and the femur rather than focus on the patella.

Prior knee braces intended to decrease anterior knee pain, specifically pain caused by mal-alignment of the patella and femur, rely on strapping or complex padding that applies a force to reposition the patella on the femur (U.S. Pat. Nos. 5,873,848; 7,749,181 B2; and U.S. Patent Application Publication 2009/0131844 A1). These braces do not function to realign the normal anatomical relationship of the tibia and femur bones and then maintain control of this normal realignment. There are braces that attempt to control rotation between the tibia and femur through designs that incorporate metal (or some other hard material) into tibia and femur components of the brace (see, for example, U.S. Pat. Nos. 4,487,200; 4,503,846; 4,624,247; and 7,666,156). Another brace identifies excessive rotation of the femur relative to the patella as the cause of knee pain and controls abnormal hip motion with a strap attached to a waist band (http://www.donjoy.com/index.asp/fuseaction/products.detail/cat/4/id/185).

There are a number of disadvantages to knee braces currently available. Besides not addressing the problems caused by rotation between the femur and tibia, most knee braces rely on bulky components that users may find ineffective, cumbersome, complex, and expensive. As noted above, even the brace that attempts to address rotation have bulky straps that do not specifically and primarily focus on realignment of the femur and tibia with a singular strap device that limits rotation and maintains the corrected rotation in a simple utilization of anchors and a single strap. Many knee braces fit the user well when first applied, but, following a small amount of activity, perspiration as well as motion from the activity itself results in movement of the brace on the skin. This movement decreases the effectiveness of the brace and requires adjustment or reapplication of the brace during activity.

A need remains for a knee brace that is simple in function and application and specifically addresses the rotation between the femur and tibia yet is lightweight, remains secure, and is easy to apply. This device also must not only correct the excessive rotation but maintain this rotational control.

All patents. patent applications, provisional patent applications and publications referred to or cited herein, are incorporated by reference in their entirety to the extent they are not inconsistent with the teachings of the specification.

### BRIEF SUMMARY OF THE INVENTION

The knee brace of the subject invention is designed to limit or decrease excessive rotation between the tibia and femur and correct resting alignment. This is accomplished by placement of two anchors, one on the thigh above the knee and one on the calf below the knee. These anchors are connected by a strap that is then tightened to correct the excessive femoral medial rotation by rotating the lower leg inwards and the thigh outwards. The strap then functions as an anti-rotational force once the correction is made. Since the degree of rotation that occurs between the tibia and femur may be small, it is important to maintain the amount of rotational control achieved by the brace. Any slippage of the brace over the skin or loosening of the strap could potentially decrease the rotational control. In order to maintain rotational control, the subject brace incorporates materials, such as silicone, that adhere the anchor to the skin, thus limiting slippage of the brace over the skin. The strap has a tightening mechanism to keep it from loosening. The subject brace first corrects the abnormal rotation of the femur over the tibia and then maintains normal joint alignment by the rotational orthosis control mechanism described.

### BRIEF DESCRIPTION OF THE SEVERAL VIEWS OF THE DRAWING

FIG. 1A is a front planar view of an upper anchor and a lower anchor of a preferred embodiment of the brace of the subject invention.

FIG. 1B is a rear planar view of the embodiment of the upper anchor and the lower anchor of the preferred embodiment of the brace shown in FIG. 1A.

FIG. 1C is a front planar view of a rotational orthosis control strap of the preferred embodiment of the brace shown in FIG. 1A.

FIG. 1D is a rear planar view of a rotational orthosis control strap of the preferred embodiment of the brace shown in FIG. 1A.

FIG. 2 is a front elevational view of a preferred embodiment of the brace of the subject invention on a knee.

FIG. 3 is an exploded view of another preferred embodiment of the brace of the subject invention.

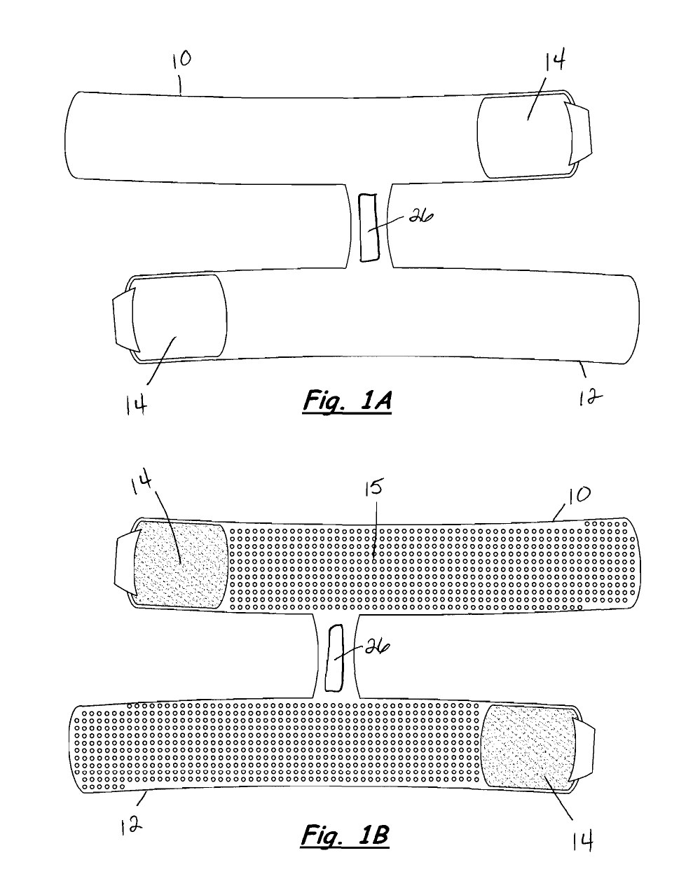

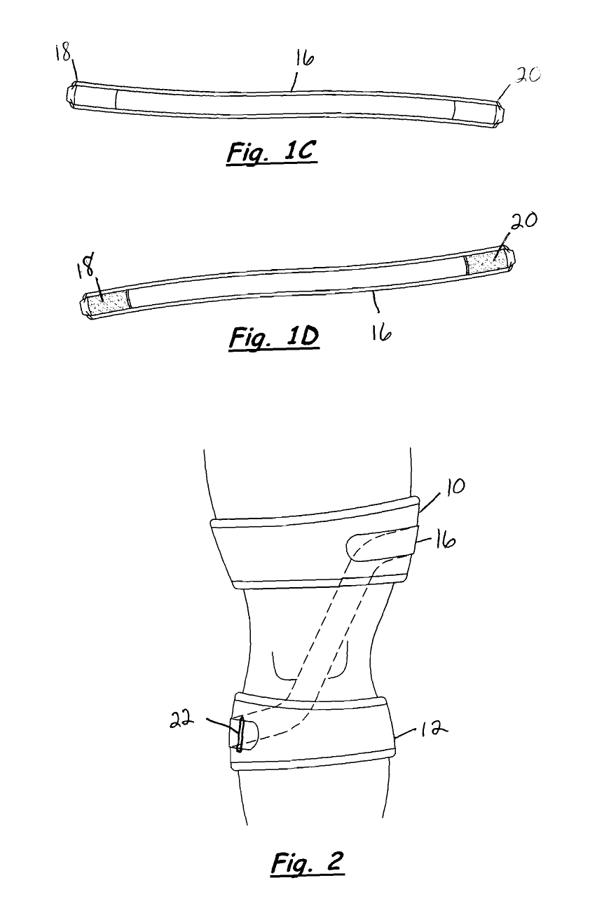

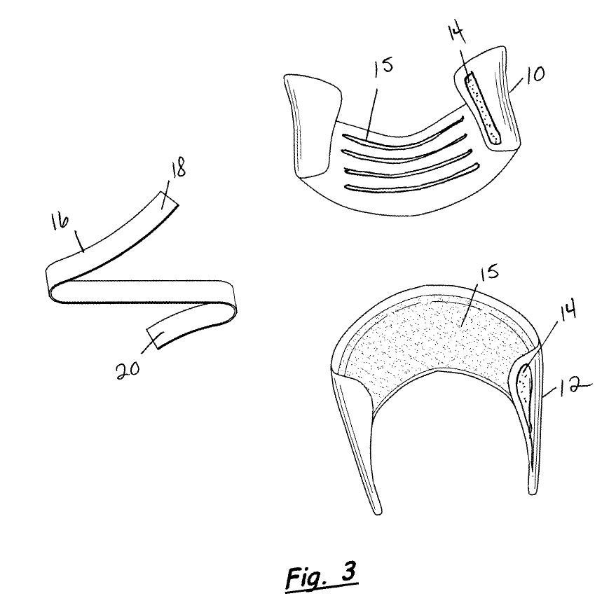

### DETAILED DESCRIPTION OF THE INVENTION

The knee brace of the subject invention comprises an upper anchor, a lower anchor, and a strap that is used to limit knee joint rotation and to alleviate knee pain.

The anchors of the subject invention capture and hold the femur and tibia of the wearer by anchoring on the thigh muscle and calf muscles. The upper anchor 10 is placed around the thigh of the wearer to control the femur. The lower anchor 12 is placed around the calf of the wearer to control the tibia. Preferably, the anchors are made of an elastomeric material to insure capture and control. Elastomeric materials suitable for the anchors of the subject invention include. but are not limited to, neoprene and urethanes. Means of securing 14 anchors to the leg of the wearer include elastic or inelastic materials secured by buckles, snaps, laces or preferably, hook and loop fastener tape for easy adjustment to any size leg.

Capture and control of femur and tibia rotation are facilitated by the anchor adhering to the skin of the wearer. The anchors comprise at least one material that has adhesive qualities with the skin. The adhesive material 15 should adhere firmly and not slip but should be able to be removed cleanly without damaging the skin. A suitable adhering material for use in the knee brace of the subject invention includes, but is not limited to, silicone and urethanes. Silicone adheres to the skin, can be removed from the skin without damage, and is non-allergenic. Certain urethanes are pliable and non-toxic to skin. The adhesion material provides a secure, non-slip anchor to which the rotational orthosis control strap is applied.

The rotational orthosis control strap 16 should have limited elasticity. A non-elastic strap is uncomfortable at the posterior knee of the wearer. Thus, although the strap should have some elasticity, elasticity must be limited so that the strap can effectively limit excessive rotation between the femur and the tibia when the brace is being worn. One end of the strap 18 is attached to the upper anchor 10 and another end of the strap 20 is attached to the lower anchor 12. The strap is applied in such a configuration to limit or decrease rotation between the femur and the tibia. Means of attachment should allow the strap to be adjusted, tightened, and secured. In a particularly preferred embodiment, a buckle 22 is provided on the lower anchor through which the strap is threaded. The wearer adjusts the strap by pulling on the end of the strap. This configuration provides the wearer leverage in tightening the strap to assure rotational control as the wearer pulls or tightens the free end of the strap up through the buckle. Other means suitable for adjusting the strap of the subject invention include, but are not limited to, rectangular rings, and incremental tightening mechanisms. Once adjusted, the end of the strap is secured to the anchor by hook and loop material.

The materials used to make the brace of the subject invention should be lightweight but durable. Since the brace must be worn firmly anchored to the skin, the materials must be such that the brace tits easily under all types of clothing. Further, since this brace is designed for use during activity, materials are preferably breathable. Air holes 24 can also be included on the anchors to increase air circulation.

In an alternative embodiment. the upper and lower anchors can be connected to one another by a central connecting attachment made of the same material as the anchors. Preferably, the bridging material has an air space 26 that fits behind the popliteal fossa of the knee joint to allow air flow to the skin and to help align proper fitting of the rotational orthosis control strap. The connection does not affect the operation of the anchors.

In use, this knee brace controls, eliminates, decreases, or prevents pain associated with a variety of conditions involving the knee joint. The brace of the subject invention maintains proper alignment of the femur and tibia by controlling rotation between these two bones. The wearer secures the upper anchor around the thigh above the knee. The lower anchor is secured around the calf below the knee. The rotational orthosis control strap connects the upper anchor to the lower anchor in a way to both limit rotation between the femur and the tibia and correct resting alignment. One skilled in the art understands how to apply the strap to limit rotation and addresses the needs of the individual wearer. For example, a preferred method for applying the strap to a wearer who pronates is to attach one end of the strap to the anterior part of the upper anchor on the front of the thigh medial to the midline of the thigh. The strap is directed lateral, around the posterior aspect of the knee and the other end of the strap is attached to the anterior portion of the lower anchor lateral to the midline of the calf. Tightening of the strap corrects the alignment causing pain and securing the strap controls and maintains the corrected position. The secured brace decreases rotation between the femur and tibia of the wearer alleviating any discomfort caused by such rotation. A tightening mechanism in the strap allows incremental orthosis control as the wearer is walking, running or exercising.

It is understood that the foregoing examples are merely illustrative of the present invention. Certain modifications of the articles and/or methods may be made and still achieve the objectives of the invention. Such modifications are contemplated as within the scope of the claimed invention.

1. A knee brace for limiting rotation of the femur of a wearer relative to the tibia of the wearer, the brace comprising:
an upper anchor capable of adhering to the skin of the wearer;
a lower anchor capable of adhering to the skin of the wearer; and
a strap comprising one end and another end; wherein the upper anchor is adhered to the skin of the wearer above the knee and the lower anchor is adhered to the skin of the wearer below the knee, one end of the strap is connected to the upper anchor and the another end of the strap is connected to the lower anchor to limit rotation of the femur of the wearer relative to the tibia of the wearer.

2. The knee brace of claim 1, wherein at least one of said upper brace and said lower anchor is configured to encircle the wearer's leg proximate the knee.

3. The knee brace of claim 1, wherein said at least one of said upper anchors and said lower anchor is elastic.

4. The knee brace of claim 1, wherein at least one of said upper brace and said lower anchor is configured to encircle the wearer's leg proximate the knee, is elastic, and further comprises means to secure said anchor to the wearer's leg.

5. The knee brace of claim 4, wherein said means to secure said anchor to the wearer's leg is selected from the group consisting of buckles, snaps, laces, and hook and loop fastener tape.

6. The knee brace of claim 1, wherein said at least one of said upper anchors and said lower anchor comprises a material selected from the group consisting of silicone and urethane polymers to adhere said anchor to the skin of the wearer.

7. The knee brace of claim 1, further comprising means to adjust said strap.

8. The knee brace of claim 7, wherein said means to adjust said strap comprises a buckle on said lower anchor.

9. The knee brace of claim 7, wherein said means to adjust said strap is rectangular rings.

10. The knee brace of claim 7, wherein said means to adjust said strap is an incremental tightening mechanism.

11. The knee brace of claim 1, wherein said upper anchor and said lower anchor are attached.

12. A method of bracing a knee to limit rotation of a femur of a wearer relative to a tibia of the wearer, the method comprising the steps of:
adhering an upper anchor to the skin of the wearer above the knee;
adhering a lower anchor to the skin of the wearer below the knee;
connecting one end of a strap to the upper anchor on the front thigh of the wearer medial the midline of the thigh;
directing the strap laterally around the posterior aspect of the knee; and
connecting another end of the strap to the lower anchor on the front calf of the wearer lateral the midline of the calf.

13. The method of claim 12, wherein at least one of said upper anchor and said lower anchor comprises a material selected from the group consisting of silicone and urethane polymers to adhere said anchor to the skin of the wearer.

14. The method of claim 12, wherein at least one of said upper anchor and said lower anchor is elastic and configured to encircle the wearer's leg and further comprises means to secure said anchor to the wearer's leg.

15. The method of claim14, wherein said means to secure said anchor to the wearer's leg is selected from the group consisting of buckles, snaps, laces, and hook and loop fastener tape.

16. The method of claim 12, wherein said strap is adjusted with a buckle on said lower anchor.

17. The method of claim 12, wherein said strap is adjusted by an incremental tightening mechanism.

18. A knee brace for limiting rotation of the femur of a wearer relative to the tibia of the wearer, the brace comprising:
an upper anchor capable of adhering to the skin of the wearer, the upper anchor is elastic and configured to encircle the wearer's thigh and further comprises means to secure said anchor to the wearer's thigh;
a lower anchor capable of adhering to the skin of the wearer, the lower anchor is elastic and configured to encircle the wearer's calf and further comprises means to secure said anchor to the wearer's calf; and
an adjustable strap comprising one end and another end; wherein the one end of the strap is connected to the upper anchor and the another end of the strap is connected to the lower anchor to correct, control and limit rotation of the femur of the wearer relative to the tibia of the wearer.

19. The knee brace of claim 18, wherein said at least one of said upper anchors and said lower anchor comprises a material selected from the group consisting of silicone and urethane polymers to adhere said anchor to the skin of the wearer.

20. The knee brace of claim 18, wherein said means to adjust said strap comprises a buckle on said lower anchor.

21. The knee brace of claim 18, wherein said strap is adjusted by an incremental tightening mechanism.

22. The knee brace of claim 18, wherein said upper anchor and said lower anchor are attached.
